(19) United States
(12) Patent Application Publication
(10) Pub. No.: US 2019/0159619 A1
(43) Pub. Date: May 30, 2019

(54) HAND SANITIZING AND SANITARY GLOVE PLACING DEVICE

(71) Applicant: Jay Burk, Fife, WA (US)

(72) Inventor: Jay Burk, Fife, WA (US)

(21) Appl. No.: 15/822,235

(22) Filed: Nov. 27, 2017

(51) Int. Cl. A47G 25/90; A61L 2/00

(52) U.S. Cl. CPC A47G 25/904; A61B 42/50; A61L 2/0082

(57) ABSTRACT

A glove dispensing device, including a plurality of buttons disposed on a front face of the glove dispensing device, a plurality of arm holes disposed on the front face of the glove dispensing device, a plurality of glove cartridges disposed within the glove dispensing device, and plurality of glove extraction devices respectively corresponding to the plurality of arm holes, the plurality of glove extraction devices to extract gloves out of the plurality of glove cartridges such that the gloves may be placed on hands of a user.

### BACKGROUND OF THE INVENTION

### 1. Field of the Invention

The present general inventive concept relates generally to a device to sanitize a user's hand and to place a sanitary glove thereupon.

### 2. Description of the Related Art

Sanitary gloves are essential accessories in the medical and food industries, particularly when medical professionals are touching patients, or when individuals are handling and/or preparing food. More specifically, bare-handed contact with food is a large cause of food-borne illnesses, and the use of gloves within healthcare facilities prevents the spreading of infections when treating patients. The practical use of gloves, in both industries, requires frequent glove-changes, which some individuals may find bothersome due to the difficulty of putting on tight sanitation gloves, especially, when one's hands are wet from washing.

Therefore, there is a need for a device that places sanitary gloves on a user's hand while minimizing the spread of dust and bacteria.

### SUMMARY

The present general inventive concept provides a device to sanitize a user's hand and to place a sanitary glove thereupon.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a glove dispensing device, including a plurality of buttons disposed on a front face of the glove dispensing device, a plurality of arm holes disposed on the front face of the glove dispensing device, a plurality of glove cartridges disposed within the glove dispensing device, and plurality of glove extraction devices respectively corresponding to the plurality of arm holes, the plurality of glove extraction devices to extract gloves out of the plurality of glove cartridges such that the gloves may be placed on hands of a user.

The glove dispensing device may further include a plurality of vertical tracks disposed within the glove dispensing device to allow the plurality of glove extraction devices to move upward and downward, and a plurality of horizontal tracks disposed within the glove dispensing device to allow the plurality of glove extraction devices to move toward and away from the plurality of glove cartridges.

The plurality of glove extraction devices may include a first blade, a second blade disposed below the first blade, a rod connected to the first blade and the second blade, at least one separation device to separate the first blade from the second blade, and a gear to move along the plurality of vertical tracks and the plurality of horizontal tracks.

At least a portion of the first blade and the second blade may be inserted into an opening of at least one of the gloves, such that the opening of the at least one of the gloves expands in response to the first blade separating from the second blade.

The glove dispensing device may further include an air sprayer to spray air into the gloves such that the gloves expand, an air compressor to generate the air, and an air distribution system to distribute the air from the air compressor to the air sprayer.

The glove dispensing device may further include a sanitizer container to contain sanitizer therein, a sanitizer sprayer system to extract the sanitizer from the sanitizer container, and a sanitizer sprayer to spray the hands of the user with sanitizer.

The glove dispensing device may further include a processor to control the sanitizer sprayer system to perform the extraction of the sanitizer from the sanitizer container, and a sensor to send a signal to the processor to perform the extraction of the sanitizer from the sanitizer container in response to the sensor sensing that the hands of the user have been inserted into the plurality of arm holes.

### BRIEF DESCRIPTION OF THE DRAWINGS

These and/or other features and utilities of the present general inventive concept will become apparent and more readily appreciated from the following description of the embodiments, taken in conjunction with the accompanying drawings of which:

FIG. 1A is a front view of a glove dispensing device, according to an exemplary embodiment of the present general inventive concept;

FIG. 1B is a side view of the glove dispensing device, according to an exemplary embodiment of the present general inventive concept;

FIG. 1C is a zoomed-in cross-sectional side view of a bottom inner portion of the glove dispensing device, according to an exemplary embodiment of the present general inventive concept;

FIG. 1D is a zoomed-in cross-sectional side view of a top inner portion of the glove dispensing device, according to an exemplary embodiment of the present general inventive concept;

FIG. 2 is a top view of the glove dispensing device, according to an exemplary embodiment of the present general inventive concept;

FIG. 3A is an angled view of a glove extraction device being lined up with one of a plurality of glove cartridges, according to an exemplary embodiment of the present general inventive concept;

FIG. 3B is an angled view of the glove extraction device manipulating a glove extracted from one of the plurality of glove cartridges, according to an exemplary embodiment of the present general inventive concept; and

FIG. 3C is an angled view of the glove extraction device after the glove is released from the glove extraction device, according to an exemplary embodiment of the present general inventive concept.

### DETAILED DESCRIPTION OF THE INVENTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

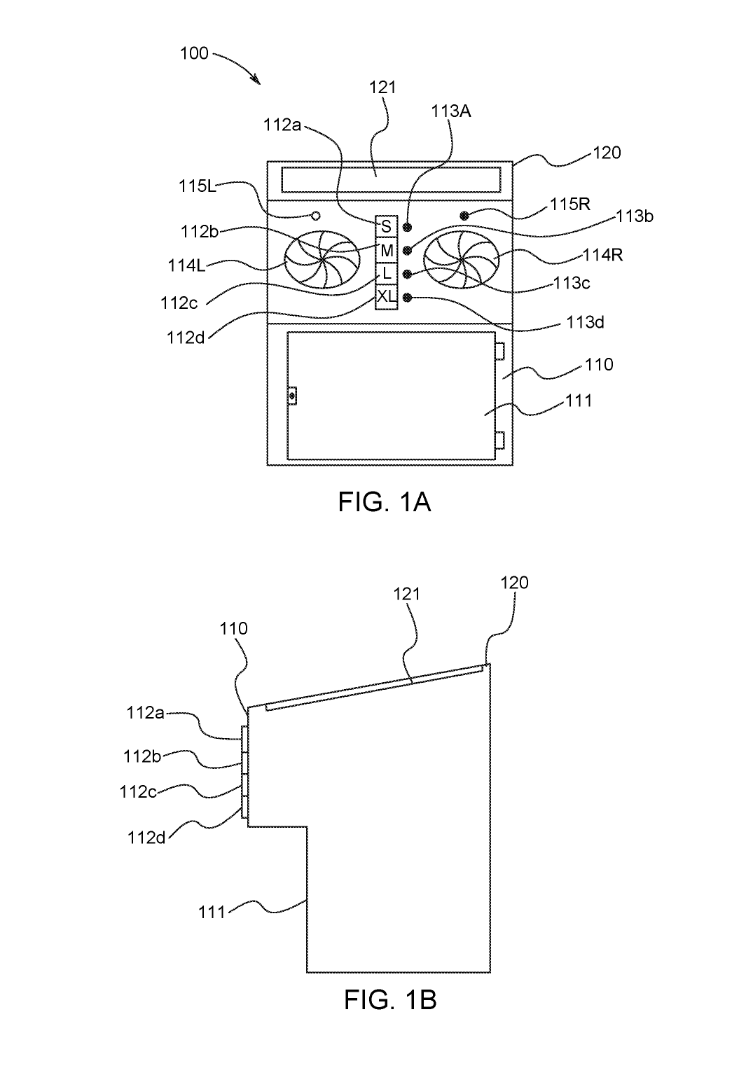

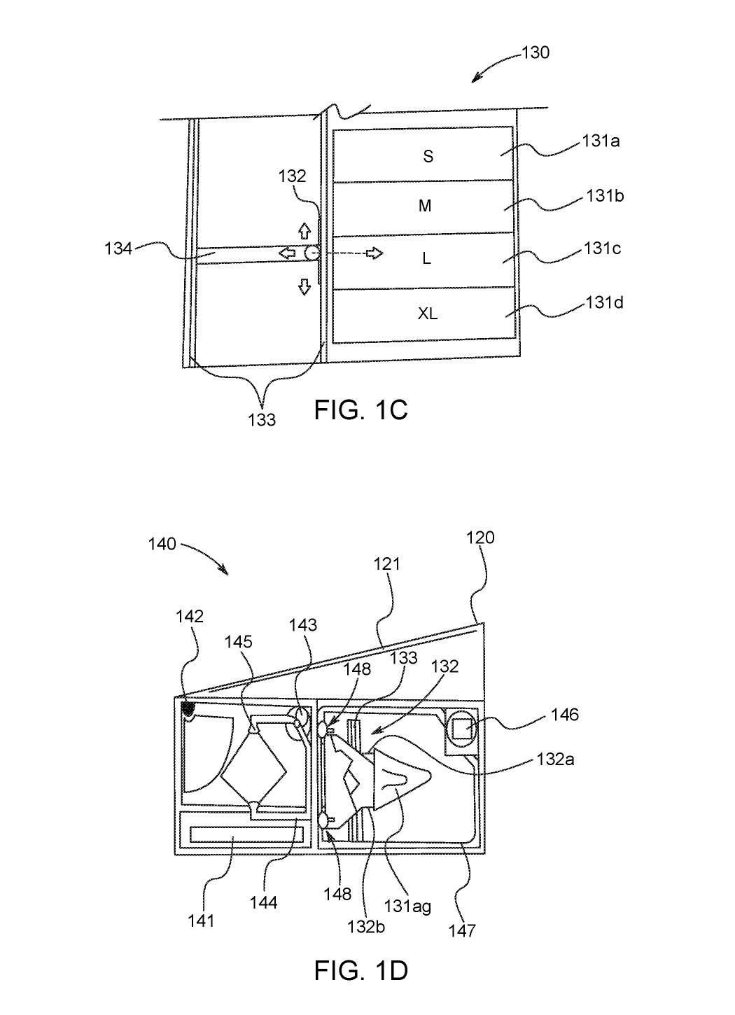

FIG. 1A is a front view of a glove dispensing device 100, according to an exemplary embodiment of the present general inventive concept.

FIG. 1B is a side view of the glove dispensing device 100, according to an exemplary embodiment of the present general inventive concept.

FIG. 1C is a zoomed-in cross-sectional side view of a bottom inner portion 130 of the glove dispensing device 100, according to an exemplary embodiment of the present general inventive concept.

FIG. 1D is a zoomed-in cross-sectional side view of a top inner portion 140 of the glove dispensing device 100, according to an exemplary embodiment of the present general inventive concept.

Referring to FIGS. 1A through 1D, the glove dispensing device 100 may include a front face portion 110, the top outer portion 120, the bottom inner portion 130, and a top inner portion 140.

Referring to FIGS. 1A and 1B, the front face portion 110 may include a door 111, a plurality of buttons 112a through 112d, a plurality of empty container indicator lights 113a through 113d, a plurality of arm holes 114L and 114R, and a plurality of ready indicator lights 115L and 115R.

The plurality of arm holes 114L and 114R may include a rubber covering that may prevent dust and bacteria from entering the plurality of arm holes 114L and 114R, and which may deform temporarily to allow for inserting of hands of the user into the plurality of arm holes 114L and 114R.

Referring to FIGS. 1A and 1B, the top outer portion 120 may include a cover 121, which may be made from a transparent material such as glass or plastic, so that a user may see inner portions of the glove dispensing device 100.

Referring to FIG. 1C, the bottom inner portion 130 may include a plurality of glove cartridges 131a through 131d, a plurality of glove extraction devices 132L and 132R, a plurality of vertical tracks 133, and a plurality of horizontal tracks 134.

The plurality of glove cartridges 131a, 131b, 1313c, and 131d may each contain one of gloves 131ag, 131bg, 131cg, and 131dg, having sizes of small, medium, large, and extra-large, respectively. The gloves 131ag, 131bg, 131cg, and 131dg may be constructed from rubber, latex, or any other type of sanitary material known to one of ordinary skill in the art.

Referring to FIG. 1D, the top inner portion 140 may include a processor 141, a sensor 142, a sanitizer container 143 containing sanitizer, a sanitizer spray system 144, a sanitizer sprayer 145, an air compressor 146, an air distribution system 147, and an air sprayer 148.

In order to replace and/or fill the sanitizer container 143 with sanitizer, the user may open the cover 121 to access the sanitizer container 143.

Referring to FIGS. 1A through 1D, the user may open the door 111 to replace at least one of the plurality of glove cartridges 131a through 131d when a respective one of the plurality of glove cartridges 131a through 131d no longer contains any gloves 131ag through 131dg. More specifically, the user may be alerted as to a need to replace one of the plurality of glove cartridges 131a through 131d, when at least one of the plurality of empty container indicator lights 113a through 113d is illuminated. In other words, the processor 141 may sense that one of the plurality of glove cartridges 131a through 131d is close to being empty, and may cause a corresponding one of the plurality of empty container indicator lights 113a through 113d to blink. When the corresponding one of the plurality of empty container indicator lights 113a through 113d ceases to blink and merely stays illuminated without blinking, this may indicate that the corresponding one of the plurality of glove cartridges 131a through 131d is completely empty.

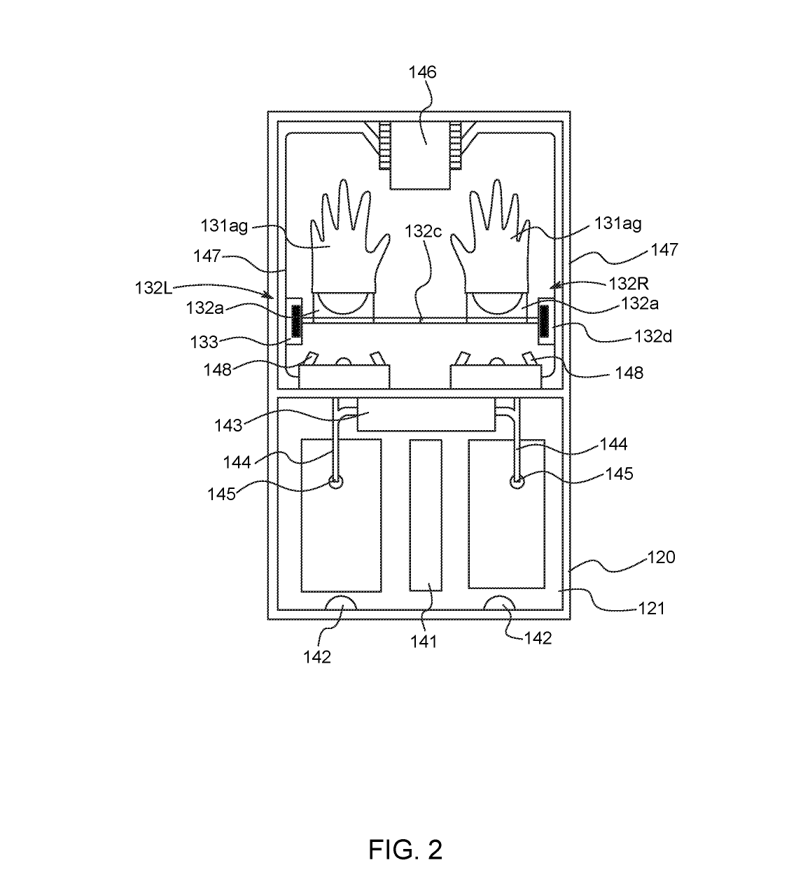

FIG. 2 is a top view of the glove dispensing device 100, according to an exemplary embodiment of the present general inventive concept.

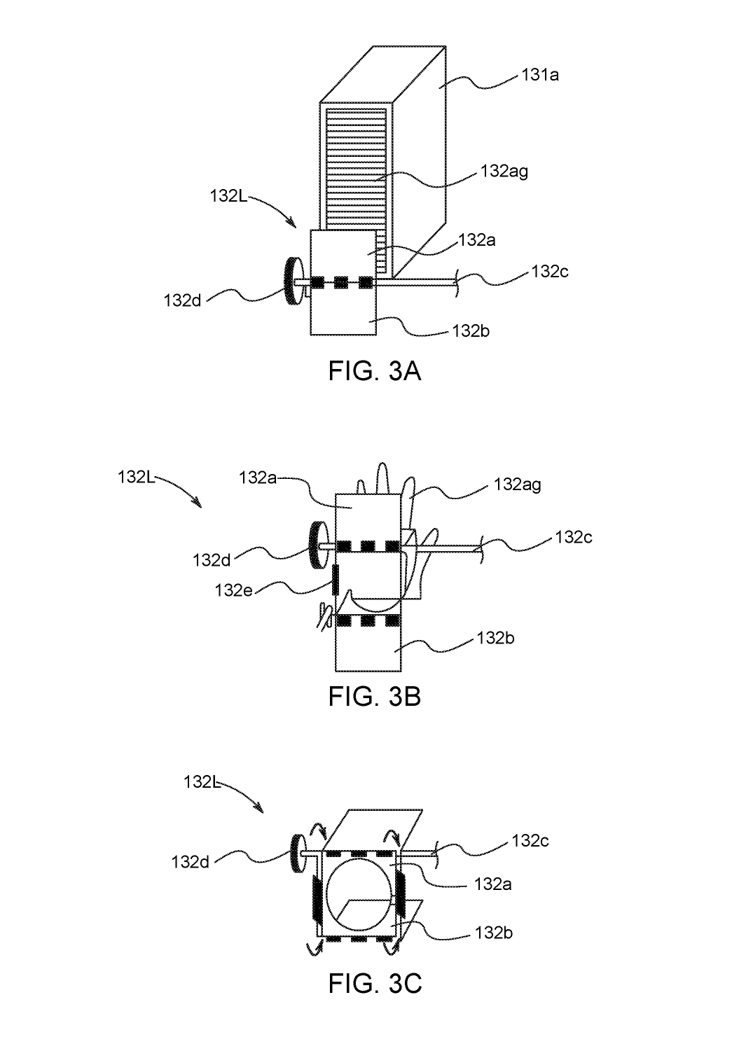

FIG. 3A is an angled view of a glove extraction device 132L being lined up with one of a plurality of glove cartridges 131a, according to an exemplary embodiment of the present general inventive concept.

FIG. 3B is an angled view of the glove extraction device 132L manipulating a glove 131ag extracted from one of the plurality of glove cartridges 131a, according to an exemplary embodiment of the present general inventive concept.

FIG. 3C is an angled view of the glove extraction device 132L after the glove 131ag is released from the glove extraction device 132L, according to an exemplary embodiment of the present general inventive concept.

The glove extraction device 132L may be the same as the glove extraction device 132R, and may include a first blade 132a, a second blade 132b, a rod 132c, a plurality of gears 132d, and at least one separation device 132e.

The rod 132c may have end portions that are inserted into grooves of the plurality of vertical tracks 133, and may have a square shape such that the first blade 132a and the second blade 132b stay in a same position while the plurality of gears 132d moves the first blade 132a, the second blade 132b, and the rod 132c up and down along the plurality of vertical tracks 133. As such, the plurality of vertical tracks 133 may also include protrusions to allow the plurality of gears 132d to climb up and down the plurality of vertical tracks 133.

The at least one separation device 132e may be embodied as a spring, piston, or any other device to allow the first blade 132a to be separated from the second blade 132b, in order to expand an opening of one of the gloves 131ag through 131dg.

Referring to FIGS. 1A through 3C, when the user desires to wear one of the gloves 131ag through 131dg, the user may press one of the plurality of buttons 112a through 112d, based on which glove-size the user desires.

After one of the plurality of buttons 112a through 112d is pressed, the glove extraction device 132L may move along the plurality of vertical tracks 133, via the plurality of gears 132d, to a base of a selected one of the plurality of glove cartridges 131a through 131d, as illustrated in FIG. 3A. FIG. 3A specifically illustrates the glove cartridge 131a, but the same method of glove extraction applies to all of the plurality of glove cartridges 131a through 131d.

The glove extraction device 132L may then move along the plurality of horizontal tracks, via the plurality of gears 132d, in a direction toward the glove cartridge 131a in order to allow the first blade 132a and the second blade 132b to enter the glove cartridge 131a, such that ends of the first blade 132a and the second blade 132b slip into an opening of the glove 131ag.

After the first blade 132a and the second blade 132b have attached thereto the glove 131ag, the glove extraction device 132L may then move along the plurality of horizontal tracks, via the plurality of gears 132d, in a direction away the glove cartridge 131a. When the glove 131ag is extracted from the glove cartridge 131a, then a spring-loaded platform may put other gloves within the glove cartridge 131a down for subsequent glove-extraction in the future.

Subsequently, the glove extraction device 132L may move along the plurality of vertical tracks 133, via the plurality of gears 132d, in a vertical direction to a position in line with the arm hole 114L.

When the glove extraction device 132L reaches the position in line with the arm hole 114L, the at least one separation device 132e may cause the first blade 132a to be separated from the second blade 132b, thereby opening up the opening of the glove 131ag.

After the glove 131ag is opened up by the separated first blade 132a and the second blade 132b, the air compressor 146 may distribute air to the air distribution system 147, and out through the air sprayer 148, thereby filling up the glove 131ag with air until the glove 131ag is expanded.

When the glove 131ag is expanded the ready indicator light 115L may illuminate to alert the user that the user may now insert a left hand into the arm hole 114L.

When the user first inserts the left hand into the arm hole 114L, the sensor 142 may sense the left hand of the user and send a signal to the processor 141 to activate the sanitizer spray system 144 to extract sanitizer from the sanitizer container 143, such that the sanitizer flows into the sanitizer spray system 144 to be expelled out from the sanitizer sprayer 145 onto a top and a bottom of the left hand of the user. As such, the left hand of the user may be thoroughly sanitized prior to entering the glove 131ag.

After the left hand of the user is sanitized, the user may continue to insert the left hand further into the glove dispensing device 100, until the glove 131ag is reached. Since the glove 131ag is already expanded due to the air being blown thereinto by the air sprayer 148, the left hand of the user easily may be inserted into the glove 131ag.

When the left hand of the user is inserted into the glove 131ag, the first blade 132a and the second blade 132b may rotate to release the glove 131ag. After the glove 131ag is released from the first blade 132a and the second blade 132b, the user may remove the left hand from the glove dispensing device 100.

The same procedure for placing a glove onto a right hand of the user may apply to the above embodiments as described for the user's left hand.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A glove dispensing device, comprising:
a plurality of buttons disposed on a front face of the glove dispensing device;
a plurality of arm holes disposed on the front face of the glove dispensing device;
a plurality of glove cartridges disposed within the glove dispensing device;
a plurality of glove extraction devices respectively corresponding to the plurality of arm holes, the plurality of glove extraction devices to extract gloves out of the plurality of glove cartridges such that the gloves may be placed on hands of a user;
a sanitizer container to contain sanitizer therein;
a sanitizer sprayer system to extract the sanitizer from the sanitizer container; and
a sanitizer sprayer to spray the hands of the user with sanitizer.

2. The glove dispensing device of claim 1, further comprising:
a plurality of vertical tracks disposed within the glove dispensing device to allow the plurality of glove extraction devices to move upward and downward; and
a plurality of horizontal tracks disposed within the glove dispensing device to allow the plurality of glove extraction devices to move toward and away from the plurality of glove cartridges.

3. The glove dispensing device of claim 2, wherein the plurality of glove extraction devices comprise:
a first blade;
a second blade disposed below the first blade;
a rod connected to the first blade and the second blade;
at least one separation device to separate the first blade from the second blade; and
at least one gear to move along the plurality of vertical tracks and the plurality of horizontal tracks.

4. The glove dispensing device of claim 3, wherein at least a portion of the first blade and the second blade are inserted into an opening of at least one of the gloves, such that the opening of the at least one of the gloves expands in response to the first blade separating from the second blade.

5. The glove dispensing device of claim 4, further comprising:
an air sprayer to spray air into the gloves such that the gloves expand;
an air compressor to generate the air; and
an air distribution system to distribute the air from the air compressor to the air sprayer.

6. (canceled)

7. The glove dispensing device of claim 1, further comprising:
a processor to control the sanitizer sprayer system to perform the extraction of the sanitizer from the sanitizer container; and
a sensor to send a signal to the processor to perform the extraction of the sanitizer from the sanitizer container in response to the sensor sensing that the hands of the user have been inserted into the plurality of arm holes.

8. A glove dispensing device, comprising:
a plurality of buttons disposed on a front face of the glove dispensing device;
a plurality of arm holes disposed on the front face of the glove dispensing device;
a plurality of glove cartridges disposed within the glove dispensing device;
a plurality of glove extraction devices respectively corresponding to the plurality of arm holes, the plurality of glove extraction devices to extract gloves out of the plurality of glove cartridges such that the gloves may be placed on hands of a user, the plurality of glove extraction devices comprising:
a first blade,
a second blade disposed below the first blade,
a rod connected to the first blade and the second blade,
at least one separation device to separate the first blade from the second blade, and
at least one gear to move along the plurality of vertical tracks and the plurality of horizontal tracks;
a plurality of vertical tracks disposed within the glove dispensing device to allow the plurality of glove extraction devices to move upward and downward;
a plurality of horizontal tracks disposed within the glove dispensing device to allow the plurality of glove extraction devices to move toward and away from the plurality of glove cartridges.
an air sprayer to spray air into the gloves such that the gloves expand;
an air compressor to generate the air; and
an air distribution system to distribute the air from the air compressor to the air sprayer,
wherein at least a portion of the first blade and the second blade are inserted into an opening of at least one of the gloves, such that the opening of the at least one of the gloves expands in response to the first blade separating from the second blade.
